(19) United States
(12) Patent Application Publication
(10) Pub. No.: US 2024/0341968 A1
(43) Pub. Date: Oct. 17, 2024

(54) EXPANDABLE EVANS WEDGE

(71) Applicant: GLOBUS MEDICAL, INC., AUDUBON, PA (US)

(72) Inventor: Richard Scheinfield, Philadelphia, PA (US)

(21) Appl. No.: 18/300,820

(22) Filed: Apr. 14, 2023

(51) Int. Cl. A61F 2/42

(52) U.S. Cl. CPC A61F 2/4202; A61F 2002/30579; A61F 2002/4223

(57) ABSTRACT

Expandable wedge implants and methods. The expandable implant has a main body, a moveable endplate pivotably connected to the main body, an actuator assembly including an actuator and two actuator pivots, and a drive assembly including a drive screw configured to move the actuator assembly to thereby expand the endplate relative to the main body. The expandable implant may be configured to correct pes planus or a flatfoot deformity using an Evans procedure during foot surgery.

### FIELD OF THE INVENTION

The present disclosure generally relates to devices and methods for correcting a bone deformity, and more particularly relates to an expandable orthopedic implant capable of being inserted into an osteotomy incision to reposition and stabilize a portion of bone.

### BACKGROUND OF THE INVENTION

In skeletally mature adults, pes planus or flatfoot deformity may be characterized by a collapse of the medial longitudinal arch of the foot, heel valgus deformity, and prominence of the medial talar bone, thereby causing the entire sole of the foot to contact or nearly contact the ground when standing. The deformity may be corrected by creating an osteotomy or single cut of the calcaneus bone of the foot and inserting a static allograft or porous titanium wedge or spacer to lengthen the lateral column of the foot.

Static wedges require the use of trials to determine the optimal wedge footprint and thickness required to correct the flatfoot deformity. In order to choose the optimal wedge thickness, multiple trials may need to be inserted and removed from the osteotomy site. This can lead to deformation of the cortical walls and increase the risk of post-operative implant subsidence into the softer cancellous bone making up a majority of the calcaneus.

For a correction requiring the use of a thicker wedge, a distraction may be necessary. The process of insertion into the distracted osteotomy site can acutely damage or break off bone on the anterior or posterior calcaneal fragments surrounding the wedge. There is potential for this damage to affect the surgeon's correction plan by forcing the surgeon to use a different wedge size than previously planned, prolonging procedure time, and/or causing excessive distraction stress on the calcaneal fragments.

There are instances where post-operative bony resorption around the implant may lead to lost correction and the need for a revision procedure to insert a thicker wedge. Revision surgery increases the risk of wound healing complications at and around the incision area, for which the soft tissues covering the lateral calcaneus are especially susceptible.

As such, there exists a need for orthopedic implants capable of being installed in an osteotomy incision to correct the flatfoot deformity, while also addressing the issues of trialing, damage to the bone fragments due to distraction, and/or revision complications.

### SUMMARY OF THE INVENTION

To meet this and other needs, implants and methods for repositioning and stabilizing a portion of bone are provided. In particular, expandable orthopedic implants, for example, for foot surgery may be used to treat pes planus or flatfoot and other patient indications. The expandable implants may be capable of continuous expansion within a thickness range, able to automatically lock at the desired thickness, and may be offered in a range of footprint options. The expandable technology allows a surgeon to dial in the wedge thickness in order to achieve the desired lengthening of the lateral column of the foot to correct the deformity.

According to one embodiment, an expandable implant includes a main body, a moveable endplate pivotably connected to the main body, the endplate defining a pair of ramps, an actuator assembly including an actuator and two actuator pivots, each actuator pivot having a ring received on the actuator and a foot with a sliding surface configured to mate with the respective ramps on the endplate, and a drive assembly configured to move the actuator assembly, wherein movement of the drive assembly translates the actuator assembly to expand the endplate away from the main body.

The expandable implant may include one or more of the following features. The endplate may be pivotably connected to the main body with a hinge pin. When expanded, the endplate may be angled relative to the main body, thereby forming a wedge shape. The main body may include a front nose, an opposite rear wall, and a pair of sidewalls connecting the front nose to the rear wall defining a central cavity. The rear wall of the main body may define a first bore configured to receive a portion of the drive assembly and a second bore in fluid communication with the central cavity. The drive assembly may include a drive screw, a lock ring, and a retaining ring aligned along a longitudinal axis. The actuator assembly may be aligned along an axis perpendicular to the longitudinal axis of the drive assembly. The drive screw may include a threaded shaft, a distal tip with a reduced diameter, and an enlarged head with a drive recess.

According to one embodiment, an expandable implant includes a main body having a front nose, an opposite rear wall, and a pair of sidewalls connecting the front nose to the rear wall defining a central cavity, a moveable endplate pivotably connected to the main body, the endplate having an outer surface with a plurality of teeth configured to engage bone and an inner surface defining a pair of ramps, an actuator assembly including an actuator having a cylindrical body and two actuator pivots, each actuator pivot having a ring received on the cylindrical body of the actuator and a foot configured to mate with one of the ramps on the endplate, and a drive assembly including a drive screw, a lock ring, and a retaining ring aligned along a longitudinal axis, wherein the drive screw is threadedly engaged with the actuator, and wherein rotation of the drive screw pulls the actuator, thereby causing the two actuator pivots to slide along the ramps of the endplate to expand the endplate away from the main body.

The expandable implant may include one or more of the following features. Each actuator pivot may have a smooth inner surface to allow each actuator pivot to rotate on the cylindrical body of the actuator. The two actuator pivots may be fitted to each end of the cylindrical body of the actuator. Each foot may define a smooth sliding surface and the ramps may define a corresponding smooth sliding surface such that the foot is configured to slide against the ramp to expand the endplate. The foot may be a male projection extending from one side of the ring, and the ramp of the endplate may be female recess configured to receive the male projection of the foot. The foot may define an arched surface at one end, and the arched surface may be receivable in a corresponding curved recess at an inner end of the female recess of the endplate. The actuator may have an enlarged cylindrical band defining a bore therethrough. The bore may be internally threaded to interface with the threaded shaft of the drive screw.

According to another embodiment, a method of correcting a bone deformity may include one or more of the following steps in any suitable order: (1) forming an osteotomy in bone, such as the calcaneus of the foot; (2) inserting an expandable implant into the osteotomy, the expandable implant having a main body, a moveable endplate pivotably connected to the main body, an actuator assembly including an actuator and two actuator pivots, each actuator pivot having a ring received on the actuator and a foot configured to mate with the endplate, and a drive assembly including a drive screw configured to move the actuator assembly; and (3) expanding the implant to correct the bone deformity by rotating the drive screw to translate the actuator assembly, thereby expanding the endplate away from the main body. The expandable implant may have an adjustable wedge thickness configured to be dialed in by a surgeon to correct the deformity, such as a flatfoot deformity. The osteotomy may be formed on a lateral aspect of the calcaneus such that expanding the implant lengthens a lateral column of the foot.

According to yet another embodiment, a method of correcting a flatfoot deformity may include one or more of the following steps in any suitable order: (1) creating an osteotomy on the lateral aspect of the calcaneus bone; (2) inserting an expandable implant into the osteotomy site; and (3) expanding the expandable implant, thereby medializing a portion of the calcaneus posterior to the osteotomy. The expandable implant does not require trialing or independent distraction prior to use, which can minimize damage and help preserve the bone.

According to yet another embodiment, a kit may include a plurality of implants of different sizes and configurations. The kit may further include one or more devices suitable for installing and/or removing the implants and systems described herein, such as insertion devices or drivers; one or more removal devices; and other tools and devices, which may be suitable for surgery.

### BRIEF DESCRIPTION OF THE DRAWINGS

The present embodiments will become more fully understood from the detailed description and the accompanying drawings, wherein:

FIG. 1 illustrates a perspective view of an expandable orthopedic implant in an expanded position according to one embodiment;

FIGS. 2A-2C show top and rear views, respectively, of the expandable orthopedic implant of FIG. 1 indicating the depth, height, and thickness of the implant;

FIG. 3 shows an exploded view of the expandable orthopedic implant of FIG. 1;

FIGS. 4A-4B show actuator pivots having sliding surfaces configured to expand the endplate according to one embodiment;

FIGS. 5A-5B show perspective views including an underside and truncated version of the ramps, respectively, of the movable endplate according to one embodiment;

FIGS. 6A-6C show side views of implant in collapsed, half-way expanded, and fully expanded positions, respectively;

FIGS. 7A-7C show the sliding surface engagement of the actuator pivot and endplate in the collapsed, half-way expanded, and fully expanded positions, respectively;

FIGS. 8A-8C show cross-sectional views of the implant in the collapsed, half-way expanded, and fully expanded positions, respectively; and

FIG. 9 shows an example of a foot anatomy and the expandable orthopedic implant of FIG. 1 implanted into an osteotomy site in the calcaneus according to one embodiment.

### DETAILED DESCRIPTION

Embodiments of the disclosure are generally directed to devices and methods for repositioning and stabilizing a portion of bone. Specifically, expandable orthopedic implants are configured to expand in thickness to properly orient the bone, stabilize the bone, and/or provide a fusion site for bone to grow. The expandable implant may be inserted into an osteotomy site and expanded to distract and stabilize the osteotomy space. The osteotomy and expanded implant may help to reshape or realign bone to relieve pain and discomfort and/or correct a deformity. For example, the expandable implant may be configured to correct pes planus or a flatfoot deformity using an Evans procedure in the calcaneus during foot surgery.

The Evans procedure may include creating an osteotomy or single cut on the lateral aspect of the calcaneus bone and inserting and expanding the expandable implant to lengthen the lateral column of the foot. The process of inserting and expanding the implant fixes the valgus heel deformity by medializing the portion of the calcaneus posterior to the osteotomy. The expandable implant allows a surgeon to dial in the wedge thickness in order to achieve the desired lengthening of the lateral column of the foot to correct the deformity.

Although generally described for use in an Evans foot procedure, it will be readily appreciated by those skilled in the art that the expandable implant may be employed in any number of suitable orthopedic approaches and procedures. The implant may be used for internal fixation of bone fractures, fusions, and/or osteotomies, including but not limited to, Cotton osteotomies, tibial osteotomies, metatarsal/cuneiform arthrodesis, as well as other indications.

Components of all of the devices disclosed herein may be manufactured of any suitable materials including metals (e.g., titanium), metal alloys (e.g., stainless steel, cobalt-chromium, and titanium alloys), ceramics, plastics, plastic composites, or polymeric materials (e.g., polyether ether ketone (PEEK), polyphenylene sulfone (PPSU), polysulfone (PSU), polycarbonate (PC), polyetherimide (PEI), polypropylene (PP), polyacetals, or mixtures or co-polymers thereof), and/or combinations thereof. In some embodiments, the devices may include radiolucent and/or radiopaque materials. The components can also be machined and/or manufactured using any suitable techniques (e.g., 3D printing).

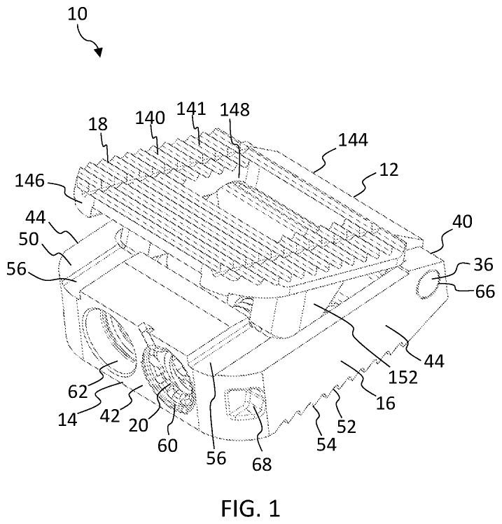

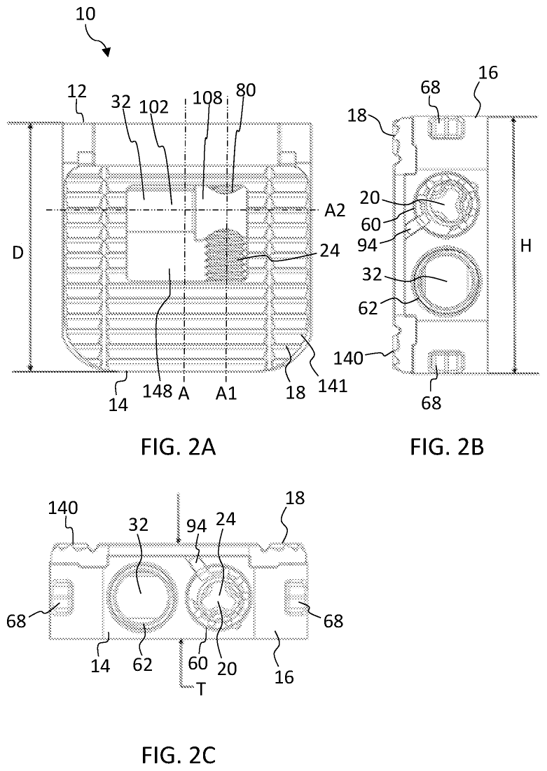

Turning now to the drawing, FIGS. 1-3 illustrate an expandable orthopedic device or implant 10 according to one embodiment. The expandable implant 10 extends along a central longitudinal axis A between front end 12 and rear end 14 of the device 10. FIG. 1 shows a perspective view of implant 10 in a fully expanded configuration. FIGS. 2A-2C show an adjustable thickness T and a footprint including a depth D and a height H of the implant 10 configured to correct the deformity. The wedge thickness T of implant 10 is adjustable to achieve the desired movement or correction of the bone(s). It should be understood that reference to the front and rear ends 12, 14, depth D, height H, and thickness T are described with respect to the direction of placement into a surgical site. For example, the front end 12 enters the surgical site first to a given depth D, oriented vertically to have a given height H, followed by the rear end 14 of the implant 10, and expanded to a given thickness T. These and other directional terms may be used herein for descriptive purposes and do not limit the orientation(s) in which the devices may be used.

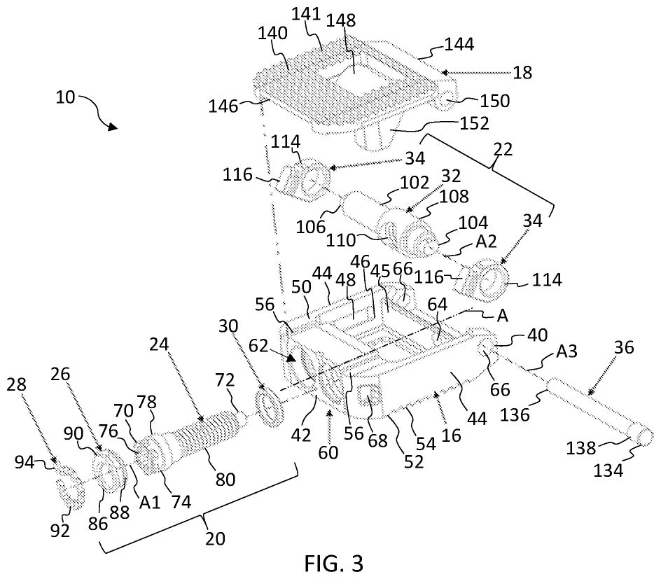

With further emphasis on the exploded view in FIG. 3, implant 10 includes a main body 16, a movable endplate 18, and a drive assembly 20 for moving an actuator assembly 22 to angulate the endplate 18. The drive assembly 20 may include a drive screw 24, a lock ring 26, a retaining ring 28, and an optional friction ring 30 aligned along longitudinal axis A1. The actuator assembly 22 may include an actuator 32 and two actuator pivots 34 aligned along perpendicular axis A2. The endplate 18 may be pivotably connected to the main body 16 with a hinge pin 36 along pivot axis A3.

The main body 16 includes a front nose 40 and an opposite rear wall 42. A pair of sidewalls 44 connect the front nose 40 to the rear wall 42. The nose 40 may be tapered or angled to facilitate insertion into the osteotomy site. The sidewalls 44 may be aligned generally in parallel to one another. The sidewalls 44 may generally form the top and bottom surfaces of implant 10 when the implant 10 is aligned vertically in the osteotomy site (as shown in FIG. 2B). A brace 45 or cross-beam may extend between the sidewalls 44 near the front nose 40 of the implant 10. The brace 45, rear wall 42, and sidewalls 44 may form a generally quadrilateral shape, such as a rectangle or square, defining a central cavity 46. An elongated recess or slot 48 may be formed along an inner portion of each sidewall 44. The slots 48 may be elongated such that the slots 48 have a given length in the direction of longitudinal axis A. The slots 48 are configured to receive the ends 104, 106 of actuator 32, and guide the actuator 32 when pulled by drive screw 24 to translate the actuator 32 along the slots 48.

The main body 16 includes an inner face 50 and an opposite outer face 52. The inner face 50 may be configured to mate with an inner surface 142 of the endplate 18 and the outer face 52 may be configured to contact bone. The inner face 50 may be defined along the rear wall 42 and sidewalls 44. The inner face 50, along rear wall 42, may define one or more notches 56 configured for receiving corresponding projections 160 from the endplate 18, when in the collapsed position. The notches 56 may extend across the inner face 50 of the rear wall 42 in the direction of the longitudinal axis A. The notches 56 may include a pair of notches aligned in parallel with longitudinal axis A and may be positioned near the sidewalls 44.

The outer face 52 of the main body 16 may include a plurality of teeth 54 or other friction increasing elements, such as ridges, roughened surfaces, keels, gripping or purchasing projections configured to retain the device 10 in bone. In one embodiment, the teeth 54 may be generally aligned with sidewalls 44 along the outer face 52. The main body 16 may be 3D printed using additive manufacturing. In this manner, the outer face 52 may be created with teeth and/or surface texturing that can better facilitate bony on-growth from the contacting cut surface of the calcaneus. The 3D printed main body 16 may allow for complex geometry to be manufactured with minimal manufacturing time.

The main body 16 defines a first bore 60 configured to receive a portion of the drive assembly 20 and a second bore 62 in fluid communication with the central cavity 46. The first and second bores 60, 62 are defined through the rear wall 42 and are laterally offset relative to the central longitudinal axis A of implant 10. The axes of the first and second bores 60, 62 may be generally parallel. The axis of first bore 60 may be aligned along longitudinal axis A1. A recess 64 may be provided in brace 45, which is configured to receive the distal tip 72 of the drive screw 24. The recess 64 may be aligned along axis Al with first bore 60. The second bore 62 allows for access to the central cavity 46, for example, to allow a graft delivery device to enter the central portion of the implant 10. Bone graft or similar bone growth inducing material may be introduced within and/or around the device 10 to further promote and facilitate bone growth.

The main body 16 further defines a pair of bores 66 configured to receive the hinge pin 36 to secure the endplate 18 to the main body 16. Bores 66 are coaxial with one another and are located in the front nose 40 of the body 16, distal to brace 45. Bores 66 are generally perpendicular to bore 60 such that the axis of each bore 66 is aligned with perpendicular axis A2. The main body 16 may define one or more instrument recesses 68 configured to be engaged by an instrument, such as an insertion instrument. For example, a pair of instrument recesses 68 may be provided on opposite sides of the body 16 near the rear wall 42 to aid in implantation.

The drive assembly 20 includes a drive screw 24, a lock ring 26, a retaining ring 28, and an optional friction ring 30 aligned along longitudinal axis A1. Longitudinal axis Al is offset laterally from central longitudinal axis A. The drive screw 24 extends from a proximal end 70 to a distal end 72. The proximal end 70 may include an enlarged head portion 74 configured to be received in bore 60 defined through the rear wall 42. The enlarged head 74 may define an instrument recess 76 configured to receive an instrument, such as a driver, to rotate or actuate the drive screw 24. The instrument recess 76 may include a tri-lobe, hex, star, or other suitable recess configured to engage with a driver instrument to apply torque to the drive screw 24. The enlarged head 74 may define a collar 78 such that a friction ring 30 may be seated beneath the collar 78. The optional friction ring 30 may be a washer or annular ring, such as a polyether ether ketone (PEEK) ring. The friction ring 30 may be assembled onto the drive screw 24, for example, below the collar 78, to increase friction or drag on the drive screw 24 during rotation.

The drive screw 24 may include a shaft with an exterior threaded portion 80 extending along its length. The distal end 72 may have a reduced distal tip, for example, having a diameter less than the diameter of the threaded shaft 80. The distal tip 72 may be smooth or unthreaded. The drive screw 24 is receivable through the bore 60 in the body 16 such that the enlarged head portion 74 of the drive screw 24 is receivable in bore 60 and the distal tip 72 is receivable in recess 64. The threaded shaft 80 threadedly engages the threaded bore 110 through the actuator 32.

The lock ring 26 may be used to eliminate rotation to the drive screw 24 while the retaining ring 28 may retain the drive screw 24 in the main body 16. The lock ring 26 may include a first ring 86 and a second ring 88 connected by a strut 90. The lock ring 26 may be shaped such that the top portion is a full ring 86 with a central through hole off center of the outer geometry. In other words, the through hole is not aligned with longitudinal axis A1. The ring 86 connects with the strut 90 to a lower C-shaped spring ring 88. The strut 90 may have a thickness greater than the thickness of the first or second rings 86, 88. The ring lock 26 is inserted onto the screw head 74 such that the C-ring 88 rests in a circumferential groove of the screw head 74 and the strut 90 is located in a notch. The top ring 86 of the lock 26 rests on a top face of the screw head 74. During insertion, the driver will pass through the offset through hole in the lock 26 and translate the lock 26 out of the groove in the screw 24. The strut 90 is translated out of the recess in the screw head 74 and is received in a recess in the main body 16. This allows the screw 24 to freely rotate with respect to the lock 26. Thus, the drive screw 24 is rotatable to thereby expand the endplate 18.

The retaining ring 28 may have a generally C-shaped body. The retaining ring 24 may include a plurality of inner and/or outer radial notches 92 that allow the ring 28 to deflect without deforming. The inner and/or outer radial notches 92 may include arcuate cutouts forming teeth. The notches 92 may be spaced equidistantly around the outer and inner surfaces of the ring 24, respectively, or otherwise configured. The retaining ring 28 may include a tab 94, which rests in a recess in the rear face of the rear wall 42, and permits the retaining ring 28 to be removed if desired. The retaining ring 28 may be placed in the bore 60 of the main body 16 behind the drive screw 24 and the lock ring 26 to prevent disassembly.

The actuator assembly 22 may include an actuator 32 and two actuator pivots 34 aligned along axis A2. Axis A2 is generally perpendicular to the longitudinal axes A, A1. The actuator 32 may include a cylindrical body 102 extending from a first end 104 to a second end 106. The first and second ends 104, 106 may define distal ends having a reduced diameter relative to the remainder of the cylindrical body 102. The ends 104, 106 are configured to be received in the respective slots 48 defined within sidewalls 44. The ends 104, 106 of actuator 32 are configured to slide or translate along slots 48 when the actuator 32 is moved by the drive screw 24.

The actuator 32 may include an enlarged cylindrical casing or band 108 proximate to one end 104, 106. Band 108 may have a diameter greater than the diameter of the rest of cylindrical body 102. The cylindrical body 102 and band 108 define a bore 110 therethrough. The bore 110 extends completely through the band 108 such that the distal tip 72 of the drive screw 24 may project beyond the actuator 32 and into recess 64 of the main body 16. The axis of the through bore 110 is aligned with the axis of bore 60 and recess 64 along axis A1. The bore 110 may be internally threaded to threadedly interface with the threaded shaft 80 of the drive screw 24. When the drive screw 24 is rotated, the threaded connection pulls the actuator 32 proximally toward rear wall 42. The actuator 32 slides or translates along slots 48, thereby causing the actuator pivots 34 to expand endplate 18.

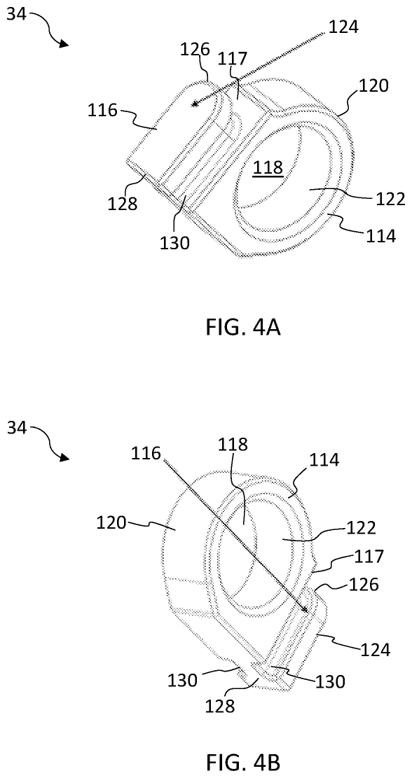

With further emphasis on FIGS. 4A-4B, each actuator pivot 34 may include a ring 114 and a foot 116. The ring 114 may be a full annular ring defining a central through hole 118. The axis of through hole 118 may be aligned with axis A2. The ring 114 may have an outer surface 120 receivable in the main body 16 and an inner surface 122 configured to contact the actuator 32. In particular, the cylindrical body 102 of the actuator 32 is positionable through the through hole 118 of each pivot actuator 34. For example, the two actuator pivots 34 may be fitted to each end of the cylindrical actuator 32. The smooth inner surface 122 of the actuator pivot 34 allows each pivot to freely rotate on the corresponding outer cylindrical surface of the actuator body 102.

The actuator pivot 34 includes a foot 116 with a sliding surface 124 configured to mate with a corresponding sliding surface 154 on the endplate 18. The foot 116 may be a male projection extending away from one side of ring 114. For example, the ring 114 may be truncated along one side to form a planar surface 117 to act as a base to support foot 116. The foot 116 may define an arched surface 126 on one end and a planar surface 128 on the opposite end. The planar surface 128 may extend along another truncated outer portion of the ring 114. The foot 116 may have a channel or groove 130 around its periphery, thereby forming the male projection, which is configured to enter a corresponding female recess 152 in the endplate 18. The groove 130 may cause portions of the foot 116, including the arched end 126, to form an overhang. The sliding surface 124 may be a smooth flat or planar surface. The sliding surfaces 124 of each pivot 34 mates with the corresponding sliding surfaces 154 on the endplate 18 to allow for sliding motion between the components. The actuator pivots 34 are able to freely rotate on the cylindrical surface of the actuator 32, which provides continuous contact between the sliding surfaces 124 of the actuator pivots 34 and the endplate 18 when the angulation between the endplate 18 and main body 16 changes.

The endplate 18 is pivotably connected to the main body 16 with a hinge pin 36. The hinge pin 36 is a cylindrical pin or rod extending from a first end 134 to a second end 136. When coupled to the main body 16, the first end 134 of the pin 36 is configured to be retained in the one bore 66 in the main body 16, the pin 36 extends through opening 150 through the endplate 18, and the second end 136 of the pin 36 is configured to be retained in the other bore 66 of the main body 16. In this manner, the endplate 18 is configured to pivot about pivot axis A3. The hinge pin 36 may have an interference 138 with the main body 16 for retention. For example, the interference area 138 may include an enlarged end receivable in bore 66. The hinge pin 36 may also have a clearance with the endplate 18 to allow for smooth rotation.

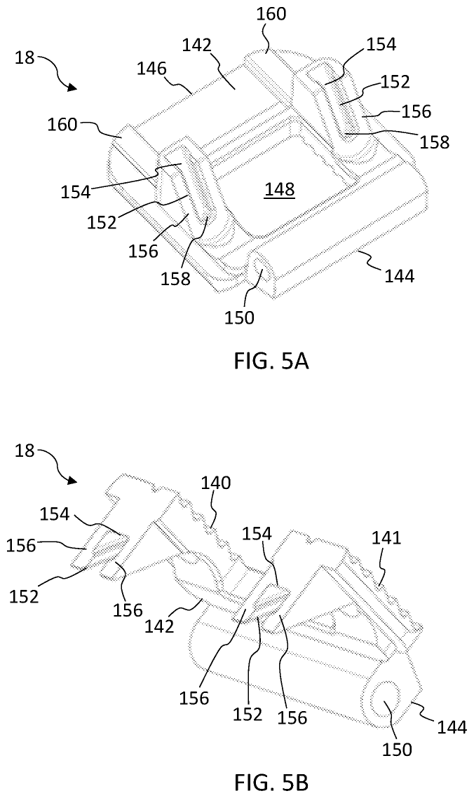

Turning now to FIGS. 5A-5B, the endplate 18 includes an outer facing surface 140 configured to interface with bone and an opposite inner facing surface 142 configured to interface with the main body 16 and actuator pivots 34. The endplate 18 includes a front portion 144 that forms part of the implant nose and an opposite rear portion 146. The outer facing portion of the nose 144 may be tapered or angled to facilitate insertion into the osteotomy site. The outer facing surface 140 of the endplate 18 may include a plurality of teeth 141 or other friction increasing elements, such as ridges, roughened surfaces, keels, gripping or purchasing projections configured to retain the device 10 in the bone. The teeth 141 may be provided along the entire outer facing surface 140 or a substantial portion thereof.

In one embodiment, the endplate 18 may be 3D printed using additive manufacturing to achieve teeth and/or surface texturing that can better facilitate bony on-growth from the contacting cut surface of the calcaneus. The 3D printed endplate may allow for complex geometry to be manufactured with minimal manufacturing time. Alternatively, the endplate 18 may be machined and optionally blasted to achieve a roughened surface. One or more openings 148 may extend through the body of the endplate 18 between the outer and inner surfaces 140, 142. In particular, a large central opening or graft window 148 may be provided through the endplate 18 and into fluid communication with the central cavity 46 of the main body 16. The graft window 148 may be open and free to receive bone-graft or other suitable bone forming material. The teeth 141 may be provided around the perimeter of the entire graft window 148.

The front portion 144 of the endplate 18 may include an elongate tube with a hollow channel 150 configured to receive a portion of hinge pin 36. The hollow channel 150 has a center axis aligned with pivot axis A3. When the hinge pin 36 is received through channel 150 and secured to main body 16, the endplate 18 is able to pivot about axis A3 in order to lift the rear end 146 of the endplate 18 relative to the main body 16. In this manner, the angle between the endplate 18 and main body 16 is able to change to a desired wedge shape and thickness.

The inner facing surface 142 includes one or more slides or ramps 152 configured to interface with the corresponding slides 124 of the actuator pivots 34. For example, a pair of slides or ramps 152 may be configured to slidably mate with the slides 124 of the respective actuator pivots 34. The pair of ramps 152 may be aligned in parallel on opposite sides of the graft window 148. The ramps 152 may be angled, diagonal, or slanted such that one end begins near inner surface 142 and extends toward rear surface 146 to terminate at a free end. Although the slides or ramps are shown in a given configuration, it will be appreciated that the slides may be sloped, slanted, or otherwise configured in any manner to provide for a desired trajectory or type of expansion.

The ramps 152 may define female channels or grooves configured to receive the mating male counterparts 116 of the actuator pivots 34. In one embodiment, the ramps 152 may be a female channel, such as a C-channel, U-channel, or J-channel. Each female slide or ramp 152 may include a sliding surface 154 bounded by a pair of legs 156. The legs 156 may define an overhang or internal flange to secure the foot 116 in the channel but still allow for slidable motion with the foot 116. An inner end of the female slide 152 may have a curved or arced recess 158 configured to receive the arched surface 126 of the foot 116. Each sliding surface 154 is configured to contact and allow for slidable engagement with the corresponding sliding surface 124 of the actuator pivot 34. It will be appreciated that the female/male configurations may be reversed or may include other suitable ramp interactions, sliding features, or mating components to provide expansion of the endplate 18.

The inner facing surface 142 may include a pair of ledges 160 receivable in corresponding notches 56 in the main body 16. Each ledge 160 may be generally aligned along the same plane as the respective ramp 152. When in the completely collapsed position, the inner facing surface 142 of the endplate 18 contacts the inner face 50 of the main body 16 and the ledges 160 are received in respective notches 56. With each respective foot 116 received within each ramp 152 of endplate 18, the sliding surfaces 124 of each actuator pivot 34 mates with the corresponding sliding surfaces 154 on the endplate 18 to allow for sliding motion between the components. The actuator pivots 34 are able to freely rotate on the cylindrical outer surface of the actuator body 102, which provides continuous contact between the sliding surface 124 of the actuator pivots 34 and the sliding surfaces 154 of the endplate 18 when the angulation between the endplate 18 and main body 16 changes. When expanded, the endplate 18 pivots about pivot axis A3, lifting endplate 18 away from main body 16, and thereby angling and increasing the wedge thickness T of implant 10.

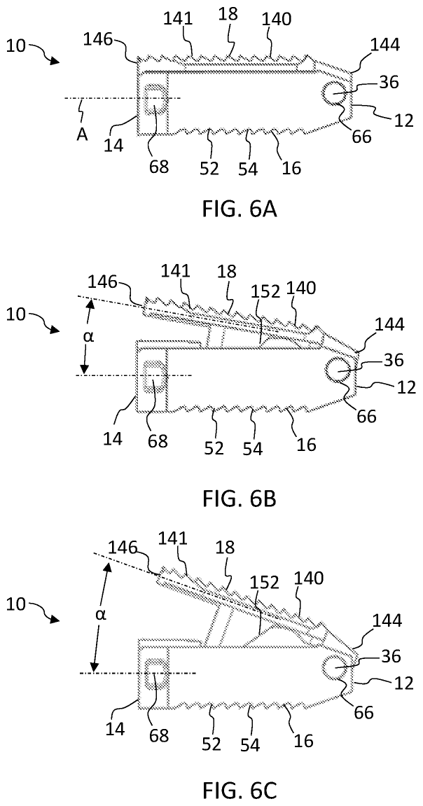

FIG. 6A show a side view of implant 10 in a collapsed position with the endplate 18 resting against the inner face 50 of the main body 16. In the collapsed position, there is no angle a between the endplate 18 and the body 16. To expand the implant 10, the drive screw 24 is rotated, which pulls the actuator 32 toward the rear wall 42 of the body 16 and causes the endplate 18 to ride up the sliding surfaces 124 on the actuator pivots 34. When this happens, the angle α of the endplate 18 relative to the body 16 changes and increases. FIG. 6B shows a side view of implant 10 with the endplate 18 partially expanded, for example, half-way expanded. In the partially expanded position, the rear end 146 of endplate 18 is expanded away from the body 16 such that a given angle α exists between the endplate 18 and the body 16. In this manner, the wedge thickness T of the implant 10 is increased to fill the osteotomy site and stabilize the bone. FIG. 6C shows a side view of implant 10 with the endplate 18 fully expanded relative to main body 16. In the fully expanded position, the rear end 146 is further expanded away from main body 16 and angle α increases, thereby providing for the greatest wedge thickness T. Due to the continuous expansions of endplate 18, it will be appreciated that the endplate 18 may be stopped at any suitable angle α and wedge thickness T for the desired surgical outcome.

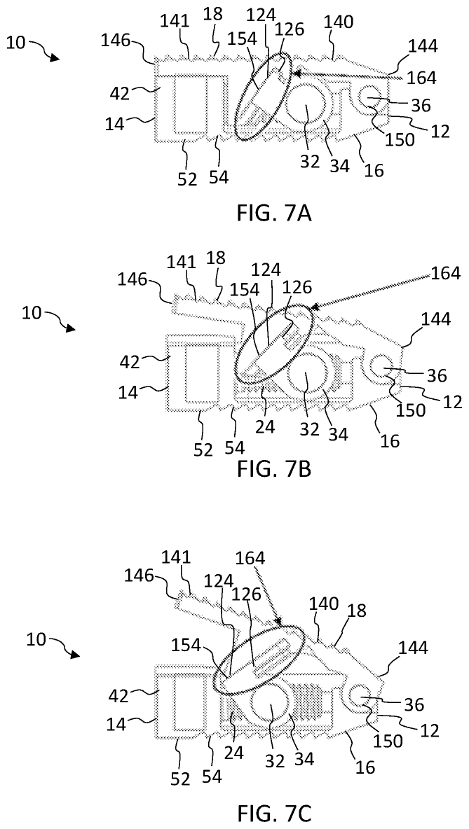

FIGS. 7A-7C show the sliding surface engagement 164 between the actuator pivot 34 and the endplate 18. In FIG. 7A, the endplate 18 is fully collapsed against the main body 16 and the sliding surface engagement 164 is shown between sliding surfaces 124, 154. In the collapsed position, the arched portion 126 of foot 116 is nested into the curved recess 158 of the ramp 152. The actuator 32 and actuator pivot 34 are located more distally toward the front 12 of the implant 10 and closer to endplate 18. As shown in FIG. 7B, endplate 18 begins to expand away from main body 16 due to the sliding surface engagement 164. As the drive screw 24 is rotated, the actuator 32 is drawn proximally along drive screw 24 and the actuator pivots 34 are also translated proximally toward rear end 14. The sliding surface engagement 164 between foot 116 and ramp 152 causes movement between sliding surfaces 124, 154 such that foot 116 travels down ramp 152, thereby expanding endplate 18 away from main body 16. FIG. 7C shows the endplate 18 fully expanded due to the sliding surface engagement 164. The drive screw 24 has pulled the actuator 32 toward rear wall 42 and actuator pivots 34 to their proximal-most position toward the rear 14 of the implant 10. The sliding surface engagement 164 between foot 116 and ramp 152 causes movement between sliding surfaces 124, 154, thereby fully expanding endplate 18 away from main body 16.

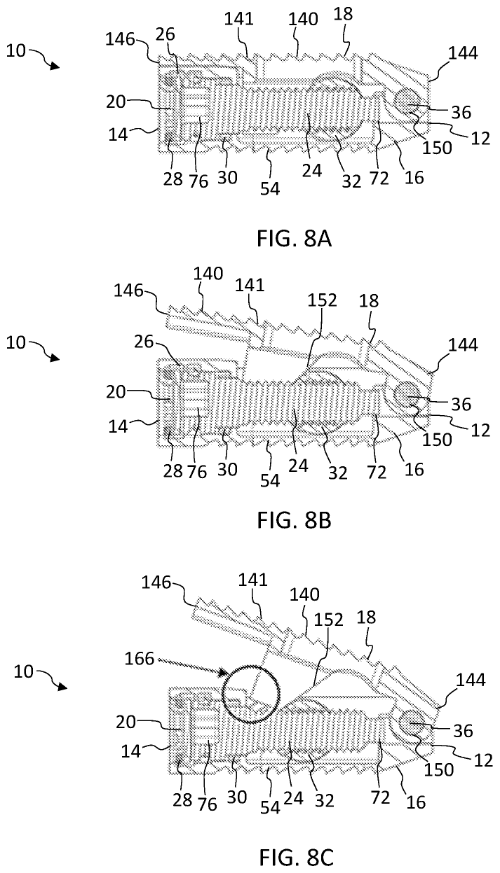

FIGS. 8A-8C show cross-sectional views of implant 10 in collapsed, partially expanded, and fully expanded configurations, respectively. FIG. 8A shows implant 10 fully collapsed with the endplate 18 resting against main body 16. The drive assembly 20 moving actuator assembly 22 causes the endplate 18 to expand and angle outward relative to main body 16. The change in angulation is driven by how far the actuator sub-assembly 22 translates in the cavity 46 of the main body 16 toward the rear 14 of the main body 16. The drive screw 24 pulls the actuator sub-assembly 22 toward the rear 14 of the implant 10, thereby expanding the endplate 18 outward and away from the main body 16. At its most expanded state shown in FIG. 8C, the pivot actuators 34 hit the rear wall 166 of the cavity 46 in the main body 16 and expansion is no longer possible.

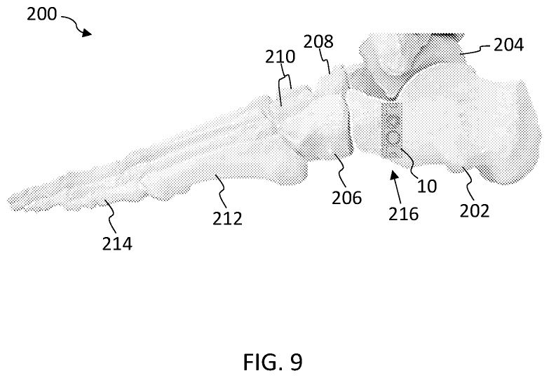

Turning now to FIG. 9, an example of a foot anatomy 200 is shown with the expandable orthopedic implant 10 implanted into an osteotomy site 216 according to one embodiment. Anatomically, the foot 200 may be divided into bones of the hindfoot, midfoot, and forefoot. The hindfoot includes the heel bone or calcaneus 202 and the ankle bone or talus 204. The midfoot includes the cuboid bone 206, the navicular bone 208, and the cuneiforms 210. The forefoot includes the metatarsal bones 212 and phalanges 214.

During an Evans foot procedure, a cut or osteotomy 216 may be made into the calcaneus bone 202. In particular, the osteotomy 216 may be formed on the lateral aspect of the calcaneus bone 202. For example, an incision may be made on the outside of the foot 200 near the front of the ankle to access the calcaneus bone 202. The osteotomy 216 may be performed behind the calcaneal cuboid joint, thereby preserving the joint for motion. Implant 10 may be positioned in the osteotomy site 216. As shown, the implant 10 may be inserted vertically with the expandable endplate 18 facing anteriorly toward the cuboid 206. It will be appreciated that the implant 10 may also be reversed such that the expandable endplate 18 faces posteriorly toward the back of the heel. The implant 10 is expanded by actuating drive screw 24, for example, with an instrument, such as a driver. By inserting and expanding the implant 10, the lateral column of the foot 200 is lengthened, pushing the front of the foot 200 into a more neutral position. This may help to fix the valgus heel deformity by medializing the portion of the calcaneus 202 posterior to the osteotomy 216. The expandable implant 10 allows a surgeon to dial in the wedge thickness in order to achieve the desired lengthening of the lateral column of the foot 200 to correct the deformity. Although described herein with respect to the Evans osteotomy, it will be appreciated that implant 10 may also be useful for Cotton osteotomies, tibial osteotomies, metatarsal/cuneiform arthrodesis, as well as other indications. It will be appreciated that the implant may also be positioned in different orientations to suit the desired surgical outcome.

Implant 10 may offer a number of advantages over a static wedge. First, the expandable wedge 10 has the ability to continuously expand over a specific range, which allows the surgeon to dial in their correction of a flatfoot deformity while performing an Evans procedure. Additionally, since the expandable wedge 10 has the capability to distract the osteotomy space while expanding, it removes the need for a secondary distracting instrument to hold open the correction at the desired final lateral column length. Second, the small starting thickness of the implant eliminates the need for trialing to determine correct implant thickness, necessary for static wedges, thereby lowering the risk of damaging walls of outer cortical shell of calcaneus that can occur when inserting/removing multiple trials. The ability to insert the expandable wedge in its collapsed state lowers risk of damage to cancellous bone that surrounds the wedge when fully implanted. Once inserted, the wedge can be gently expanded to avoid bone damage. Thirdly, if revision surgery is necessary to adjust the wedge thickness to address lost correction due to bony resorption around the implant, a stab incision may be used to engage the driver with the implant rather than having to remove an entire static wedge and insert a larger one.

Although the invention has been described in detail and with reference to specific embodiments, it will be apparent to one skilled in the art that various changes and modifications can be made without departing from the spirit and scope of the invention. Thus, it is intended that the invention covers the modifications and variations of this invention provided they come within the scope of the appended claims and their equivalents. It is expressly intended, for example, that all components of the various devices disclosed above may be combined or modified in any suitable configuration.

What is claimed is:

1. A method for stabilizing a An expandable implant comprising:
a main body;
a moveable endplate pivotably connected to the main body, the endplate defining a pair of ramps;
an actuator assembly including an actuator and two actuator pivots, each actuator pivot having a ring received on the actuator and a foot with a sliding surface configured to mate with respective ramps on the endplate; and
a drive assembly configured to move the actuator assembly, wherein movement of the drive assembly translates the actuator assembly to expand the endplate away from the main body.

2. The expandable implant of claim 1, wherein the endplate is pivotably connected to the main body with a hinge pin.

3. The expandable implant of claim 1, wherein when expanded, the endplate is angled relative to the main body.

4. The expandable implant of claim 1, wherein the main body includes a front nose, an opposite rear wall, and a pair of sidewalls connecting the front nose to the rear wall defining a central cavity.

5. The expandable implant of claim 4, wherein the rear wall of the main body defines a first bore configured to receive a portion of the drive assembly and a second bore in fluid communication with the central cavity.

6. The expandable implant of claim 1, wherein the drive assembly includes a drive screw, a lock ring, and a retaining ring aligned along a longitudinal axis.

7. The expandable implant of claim 6, wherein the actuator assembly is aligned along an axis perpendicular to the longitudinal axis of the drive assembly.

8. The expandable implant of claim 6, wherein the drive screw includes a threaded shaft, a distal tip with a reduced diameter, and an enlarged head with a drive recess.

9. An expandable implant comprising:
a main body having a front nose, an opposite rear wall, and a pair of sidewalls connecting the front nose to the rear wall defining a central cavity;
a moveable endplate pivotably connected to the main body, the endplate having an outer surface with a plurality of teeth configured to engage bone and an inner surface defining a pair of ramps;
an actuator assembly including an actuator having a cylindrical body and two actuator pivots, each actuator pivot having a ring received on the cylindrical body of the actuator and a foot configured to mate with one of the ramps on the endplate; and
a drive assembly including a drive screw, a lock ring, and a retaining ring aligned along a longitudinal axis, wherein the drive screw is threadedly engaged with the actuator, and wherein rotation of the drive screw pulls the actuator, thereby causing the two actuator pivots to slide along the ramps of the endplate to expand the endplate away from the main body.

10. The expandable implant of claim 9, wherein each actuator pivot has a smooth inner surface to allow each actuator pivot to rotate on the cylindrical body of the actuator.

11. The expandable implant of claim 9, wherein the two actuator pivots are fitted to each end of the cylindrical body of the actuator.

12. The expandable implant of claim 9, wherein each foot defines a smooth sliding surface and the ramps define a corresponding smooth sliding surface such that the foot is configured to slide against the ramp to expand the endplate.

13. The expandable implant of claim 9, wherein the foot is a male projection extending from one side of the ring, and the ramp of the endplate is female recess configured to receive the male projection of the foot.

14. The expandable implant of claim 13, wherein the foot defines an arched surface at one end, and the arched surface is receivable in a corresponding curved recess at an inner end of the female recess of the endplate.

15. The expandable implant of claim 9, wherein the actuator has an enlarged cylindrical band defining a bore therethrough, wherein the bore is internally threaded to interface with the threaded shaft of the drive screw.
